(19) United States
(12) Patent Application Publication
(10) Pub. No.: US 2024/0342498 A1
(43) Pub. Date: Oct. 17, 2024

(54) REMOTE DRIVE TECHNIQUES FOR MAGNETIC FIELD THERAPY

(71) Applicant: Wave Neuroscience, Inc., Newport Beach, CA (US)

(72) Inventor: James William Phillips, Fountain Valley, CA (US)

(21) Appl. No.: 18/638,206

(22) Filed: Apr. 17, 2024

Related U.S. Application Data
(60) Provisional application No. 63/496,500, filed on Apr. 17, 2023.

(51) Int. Cl. A61N 2/00; A61N 2/02

(52) U.S. Cl. CPC A61N 2/006; A61N 2/02

(57) ABSTRACT

A system for providing treatment to a subject including a first magnetic source, a motor coupled to the first magnetic source, a second magnetic source positioned in proximity to the first magnetic source, at least one memory storing computer-executable instructions, and at least one processor for executing the instructions stored on the memory. Execution of the instructions causes the at least one processor to, when the second magnetic source is positioned in proximity to a head of the subject, operate the motor to rotate the first magnetic source causing the first magnetic source to produce a first magnetic field, wherein the first magnetic field is applied to the second magnetic source and causes the second magnetic source to rotate and produce a second magnetic field that is applied to the head of the subject to provide a therapeutic treatment.

### CROSS-REFERENCE TO RELATED APPLICATIONS

This application claims priority to and the benefit of U.S. Provisional Patent Application No. 63/496,500, titled “REMOTE DRIVE TECHNIQUES FOR MAGNETIC FIELD THERAPY” and filed on Apr. 17, 2023, the entire contents of which is hereby incorporated by reference herein.

### FIELD

The disclosure relates to systems and methods that utilize remote drive techniques to provide magnetic field therapy.

### BACKGROUND

Magnetic field therapy systems are used to provide therapeutic treatment. Magnetic field pulses or waves stimulate nerve cells and neuronal circuitry in the brain to improve certain mental disorders such as schizophrenia, obsessive compulsive disorder (OCD), depression, and others. In some cases, alternating magnetic fields are generated by a moving magnet (or magnets) and administered to the subject. Magnetic field therapy is generally considered a “whole brain” treatment and the exact location of the magnet(s) over the subject's scalp can vary from treatment-to-treatment or subject-to-subject. In some cases, optimal stimulation is provided by a number of permanent magnets all moved rhythmically over the subject's scalp. Each permanent magnet is moved using a motor. Alternatively, the magnets are physically connected to each other with a shaft or belt, so that the magnets move synchronously, using only one motor. However, these components (e.g., motors, belts, etc.) can increase the weight, cost, and size of such therapy systems (or devices).

### SUMMARY

The disclosure relates generally to systems and methods that utilize remote drive techniques to provide magnetic field therapy.

It is to be understood that any combination of features from the methods disclosed herein and/or from the systems and/or devices disclosed herein may be used together, and/or that any features from any or all of these aspects may be combined with any of the features of the embodiments and/or examples disclosed herein to achieve the benefits as described in this disclosure.

At least one aspect of the present disclosure is directed to a system for providing treatment to a subject. The system includes a first magnetic source, a motor coupled to the first magnetic source, a second magnetic source positioned in proximity to the first magnetic source, at least one memory storing computer-executable instructions, and at least one processor for executing the instructions stored on the memory. Execution of the instructions causes the at least one processor to, when the second magnetic source is positioned in proximity to a head of the subject, operate the motor to rotate the first magnetic source causing the first magnetic source to produce a first magnetic field, wherein the first magnetic field is applied to the second magnetic source and causes the second magnetic source to rotate and produce a second magnetic field that is applied to the head of the subject to provide a therapeutic treatment.

In some embodiments, the therapeutic treatment includes transcranial magnetic stimulation (TMS). In some embodiments, the first magnetic field and the second magnetic field are applied to the head of the subject to provide the therapeutic treatment. In some embodiments, the first magnetic source and the second magnetic source are permanent magnets. In some embodiments, the first magnetic source and the second magnetic source are each cylindrical magnets having a height and a diameter. In some embodiments, the motor is configured to rotate the first magnetic source in a first direction about an axis parallel to the height of the first magnetic source. In some embodiments, the second magnetic source is positioned such that the first magnetic field causes the second magnetic source to rotate in a second direction, the second direction being opposite from the first direction. In some embodiments, the height of the second magnetic source is parallel to the height of the first magnetic source. In some embodiments, the second magnetic source is positioned such that the first magnetic field causes the second magnetic source to rotate in the first direction. In some embodiments, an end of the second magnetic source faces an end of the first magnetic source, the end of the second magnetic source corresponding to a surface having the diameter of the second magnetic source and the end of the first magnetic source corresponding to a surface having the diameter of the first magnetic source.

In some embodiments, the system includes a third magnetic source positioned in proximity to the second magnetic source, wherein the second magnetic field is applied to the third magnetic source and causes the third magnetic source to rotate and produce a third magnetic field. In some embodiments, the third magnetic source is positioned such that the second magnetic field causes the third magnetic source to rotate in the first direction. In some embodiments, the third magnetic source is a cylindrical magnet having a height and a diameter. In some embodiments, the height of the third magnetic source is parallel to the height of the second magnetic source. In some embodiments, an end of the third magnetic source faces an end of the second magnetic source, the end of the third magnetic source corresponding to a surface having the diameter of the third magnetic source and the end of the second magnetic source corresponding to a surface having the diameter of the second magnetic source.

In some embodiments, the motor is configured to rotate the first magnetic source at a first frequency. In some embodiments, the rotation of the first magnetic source at the first frequency causes the first magnetic field to have a second frequency. In some embodiments, the first magnetic field, when applied to the second magnetic source, causes the second magnetic source to rotate at a third frequency. In some embodiments, the rotation of the second magnetic source at the third frequency causes the second magnetic field to have a fourth frequency.

Another aspect of the disclosure is directed to a method of treating a subject. The method includes providing a first magnetic source, a motor coupled to the first magnetic source, and a second magnetic source, the second magnetic source being positioned in proximity to the first magnetic source. The second magnetic source is positioned in proximity to a head of the subject. The motor is operated to rotate the first magnetic source causing the first magnetic source to produce a first magnetic field. The first magnetic field is applied to the second magnetic source and causes the second magnetic source to rotate and produce a second magnetic field that is applied to the head of the subject to provide a therapeutic treatment.

In some embodiments, the therapeutic treatment includes transcranial magnetic stimulation (TMS). In some embodiments, the method includes positioning the first magnetic source in proximity to the head of the subject, wherein the first magnetic field and the second magnetic field are applied to the head of the subject to provide the therapeutic treatment. In some embodiments, the first magnetic source and the second magnetic source are permanent magnets. In some embodiments, the first magnetic source and the second magnetic source are each cylindrical magnets having a height and a diameter. In some embodiments, operating the motor to rotate the first magnetic source includes rotating the first magnetic source in a first direction about an axis parallel to the height of the first magnetic source. In some embodiments, the second magnetic source is positioned such that the first magnetic field causes the second magnetic source to rotate in a second direction, the second direction being opposite from the first direction. In some embodiments, the height of the second magnetic source is parallel to the height of the first magnetic source. In some embodiments, the second magnetic source is positioned such that the first magnetic field causes the second magnetic source to rotate in the first direction. In some embodiments, an end of the second magnetic source faces an end of the first magnetic source, the end of the second magnetic source corresponding to a surface having the diameter of the second magnetic source and the end of the first magnetic source corresponding to a surface having the diameter of the first magnetic source.

In some embodiments, the method includes providing a third magnetic source positioned in proximity to the second magnetic source and applying the second magnetic field to the third magnetic source and causing the third magnetic source to rotate and produce a third magnetic field. In some embodiments, the third magnetic source is positioned such that the second magnetic field causes the third magnetic source to rotate in the first direction. In some embodiments, the third magnetic source is a cylindrical magnet having a height and a diameter. In some embodiments, the height of the third magnetic source is parallel to the height of the second magnetic source. In some embodiments, an end of the third magnetic source faces an end of the second magnetic source, the end of the third magnetic source corresponding to a surface having the diameter of the third magnetic source and the end of the second magnetic source corresponding to a surface having the diameter of the second magnetic source.

In some embodiments, operating the motor to rotate the first magnetic source includes rotating the first magnetic source at a first frequency. In some embodiments, the rotation of the first magnetic source at the first frequency causes the first magnetic field to have a second frequency. In some embodiments, the first magnetic field, when applied to the second magnetic source, causes the second magnetic source to rotate at a third frequency. In some embodiments, the rotation of the second magnetic source at the third frequency causes the second magnetic field to have a fourth frequency.

Another aspect of the present disclosure is directed to a system for providing treatment to a subject. The system includes a first magnetic source, a second magnetic source positioned in proximity to the first magnetic source, at least one memory storing computer-executable instructions, and at least one processor for executing the instructions stored on the memory. Execution of the instructions causes the at least one processor to, when in proximity to a head of the subject, control the first magnetic source to produce a first magnetic field, wherein the first magnetic field is applied to the second magnetic source and causes the second magnetic source to rotate and produce a second magnetic field that is applied to the head of the subject to provide a therapeutic treatment.

In some embodiments, the first magnetic source is a stationary magnetic source. In some embodiments, the first magnetic source is an electromagnetic coil. In some embodiments, controlling the first magnetic source to produce the first magnetic field includes providing a current to the first magnetic source to produce the first magnetic field. In some embodiments, the therapeutic treatment includes transcranial magnetic stimulation (TMS). In some embodiments, the first magnetic field and the second magnetic field are applied to the head of the subject to provide the therapeutic treatment. In some embodiments, the second magnetic source is a permanent magnet. In some embodiments, the second magnetic source is a cylindrical magnet having a height and a diameter. In some embodiments, the second magnetic source is positioned such that the first magnetic field causes the second magnetic source to rotate in a first direction.

In some embodiments, the system includes a third magnetic source positioned in proximity to the second magnetic source, wherein the second magnetic field is applied to the third magnetic source and causes the third magnetic source to rotate and produce a third magnetic field. In some embodiments, the third magnetic source is positioned such that the second magnetic field causes the third magnetic source to rotate in a second direction, the second direction being opposite from the first direction. In some embodiments, the third magnetic source is a cylindrical magnet having a height and a diameter. In some embodiments, the height of the third magnetic source is parallel to the height of the second magnetic source. In some embodiments, an end of the third magnetic source faces an end of the second magnetic source, the end of the third magnetic source corresponding to a surface having the diameter of the third magnetic source and the end of the second magnetic source corresponding to a surface having the diameter of the second magnetic source.

Another aspect of the present disclosure is directed to a method of treating a subject. The method includes providing a first magnetic source and a second magnetic source positioned in proximity to the first magnetic source. The second magnetic source is positioned in proximity to a head of the subject. The first magnetic source is controlled to produce a first magnetic field. The first magnetic field is applied to the second magnetic source and causes the second magnetic source to rotate and produce a second magnetic field that is applied to the head of the subject to provide a therapeutic treatment.

In some embodiments, the first magnetic source is a stationary magnetic source. In some embodiments, the first magnetic source is an electromagnetic coil. In some embodiments, controlling the first magnetic source to produce the first magnetic field includes providing a current to the first magnetic source to produce the first magnetic field. In some embodiments, the therapeutic treatment includes transcranial magnetic stimulation (TMS). In some embodiments, the method includes positioning the first magnetic source in proximity to the head of the subject, wherein the first magnetic field and the second magnetic field are applied to the head of the subject to provide the therapeutic treatment. In some embodiments, the second magnetic source is a permanent magnet. In some embodiments, the second magnetic source is a cylindrical magnet having a height and a diameter. In some embodiments, the second magnetic source is positioned such that the first magnetic field causes the second magnetic source to rotate in a first direction.

In some embodiments, the method includes providing a third magnetic source positioned in proximity to the second magnetic source and applying the second magnetic field to the third magnetic source and causing the third magnetic source to rotate and produce a third magnetic field. In some embodiments, the third magnetic source is positioned such that the second magnetic field causes the third magnetic source to rotate in a second direction, the second direction being opposite from the first direction. In some embodiments, the third magnetic source is a cylindrical magnet having a height and a diameter. In some embodiments, the height of the third magnetic source is parallel to the height of the second magnetic source. In some embodiments, an end of the third magnetic source faces an end of the second magnetic source, the end of the third magnetic source corresponding to a surface having the diameter of the third magnetic source and the end of the second magnetic source corresponding to a surface having the diameter of the second magnetic source.

Another aspect of the present disclosure is directed to a wearable device for providing treatment to a subject. The device includes a frame configured to be disposed on a head of the subject, a first magnetic source positioned within the frame, a motor coupled to the first magnetic source, a second magnetic source positioned within the frame and in proximity to the first magnetic source, at least one memory storing computer-executable instructions, and at least one processor for executing the instructions stored on the memory. Execution of the instructions causes the at least one processor to, when in proximity to a head of the subject, operate the motor to rotate the first magnetic source causing the first magnetic source to produce a first magnetic field, wherein the first magnetic field is applied to the second magnetic source and causes the second magnetic source to rotate and produce a second magnetic field that is applied to the head of the subject to provide a therapeutic treatment. In some embodiments, the first magnetic source and the second magnetic source are each cylindrical magnets having a height and a diameter.

In some embodiments, the device includes a first shaft running through the first magnetic source parallel to the height of the first magnetic source and a second shaft running through the second magnetic source parallel to the height of the second magnetic source. In some embodiments, the first shaft and the second shaft are both coupled to the frame and configured to rotate with respect to the frame. In some embodiments, the first shaft is coupled to the first magnetic source such that rotation of the first shaft causes the first magnetic source to rotate and the second shaft is coupled to the second magnetic source such that rotation of the second shaft causes the second magnetic source to rotate. In some embodiments, the motor is coupled to the first shaft and configured to rotate the first magnetic source by rotating the first shaft.

In some embodiments, the motor is configured to rotate the first magnetic source in a first direction about an axis parallel to the height of the first magnetic source. In some embodiments, the second magnetic source is positioned such that the first magnetic field causes the second magnetic source to rotate in a second direction, the second direction being opposite from the first direction. In some embodiments, the height of the second magnetic source is adjacent to the height of the first magnetic source. In some embodiments, the second magnetic source is positioned such that the first magnetic field causes the second magnetic source to rotate in the first direction. In some embodiments, an end of the second magnetic source faces an end of the first magnetic source, the end of the second magnetic source corresponding to a surface having the diameter of the second magnetic source and the end of the first magnetic source corresponding to a surface having the diameter of the first magnetic source.

In some embodiments, the device includes a third magnetic source positioned within the frame and in proximity to the second magnetic source, wherein the second magnetic field is applied to the third magnetic source and causes the third magnetic source to rotate and produce a third magnetic field. In some embodiments, the third magnetic source is positioned such that the second magnetic field causes the third magnetic source to rotate in the first direction. In some embodiments, the third magnetic source is a cylindrical magnet having a height and a diameter. In some embodiments, the device includes a third shaft running through the third magnetic source parallel to the height of the third magnetic source. In some embodiments, the third shaft is coupled to the frame and configured to rotate with respect to the frame. In some embodiments, the third shaft is coupled to the third magnetic source such that rotation of the third shaft causes the third magnetic source to rotate. In some embodiments, the height of the third magnetic source is adjacent to the height of the second magnetic source. In some embodiments, an end of the third magnetic source faces an end of the second magnetic source, the end of the third magnetic source corresponding to a surface having the diameter of the third magnetic source and the end of the second magnetic source corresponding to a surface having the diameter of the second magnetic source.

In some embodiments, the at least one memory and the at least one processor are disposed within the frame. In some embodiments, the device includes a cover coupled to the frame and disposed between the first and second magnetic sources and the head of the subject. In some embodiments, the frame includes a curvature that enables the frame to rest on the head of the subject.

Another aspect of the present disclosure is directed to a system for providing treatment to a subject. The system includes a base station including a first magnetic source, a frame configured to be disposed on a head of the subject, a second magnetic source positioned within the frame, at least one memory storing computer-executable instructions, and at least one processor for executing the instructions stored on the memory. Execution of the instructions causes the at least one processor to, when in proximity to a head of the subject, control the first magnetic source to produce a first magnetic field, wherein the first magnetic field is applied to the second magnetic source and causes the second magnetic source to rotate and produce a second magnetic field that is applied to the head of the subject to provide a therapeutic treatment.

In some embodiments, the first magnetic field causes the second magnetic source to rotate and produce the second magnetic field when the frame is positioned adjacent to the base station. In some embodiments, the first magnetic source is a stationary magnetic source. In some embodiments, the first magnetic source is an electromagnetic coil. In some embodiments, controlling the first magnetic source to produce the first magnetic field includes providing a current to the first magnetic to produce the first magnetic field. In some embodiments, the therapeutic treatment includes transcranial magnetic stimulation (TMS). In some embodiments, the first magnetic source and the second magnetic source are permanent magnets. In some embodiments, the first magnetic source and the second magnetic source are each cylindrical magnets having a height and a diameter. In some embodiments, the base station includes a motor coupled to the first magnetic source. In some embodiments, controlling the first magnetic source to produce the first magnetic field includes operating the motor to rotate the first magnetic source. In some embodiments, the motor is configured to rotate the first magnetic source about an axis parallel to the height of the first magnetic source. In some embodiments, the at least one processor is configured to operate the motor. In some embodiments, the system includes a third magnetic source positioned within the frame and in proximity to the second magnetic source, wherein the second magnetic field is applied to the third magnetic source and causes the third magnetic source to rotate and produce a third magnetic field.

Another aspect of the present disclosure is directed to a method of treating a subject. The method includes providing a base station including a first magnetic source and providing a frame including a second magnetic source. The frame is disposed on the head of the subject. The second magnetic source of the frame is positioned in proximity to the first magnetic source of the base station. The first magnetic source is controlled to produce a first magnetic field. The first magnetic field is applied to the second magnetic source and causes the second magnetic source to rotate and produce a second magnetic field that is applied to the head of the subject to provide a therapeutic treatment.

In some embodiments, positioning the second magnetic source of the frame in proximity to the first magnetic source of the base station includes positioning the frame to be adjacent to the base station. In some embodiments, the first magnetic source is a stationary magnetic source. In some embodiments, the first magnetic source is an electromagnetic coil. In some embodiments, controlling the first magnetic source to produce the first magnetic field includes providing a current to the first magnetic to produce the first magnetic field. In some embodiments, the therapeutic treatment includes transcranial magnetic stimulation (TMS). In some examples, the first magnetic source and the second magnetic source are permanent magnets. In some examples, the first magnetic source and the second magnetic source are each cylindrical magnets having a height and a diameter. In some embodiments, the base station includes a motor coupled to the first magnetic source. In some embodiments, controlling the first magnetic source to produce the first magnetic field includes operating the motor to rotate the first magnetic source. In some embodiments, the motor is configured to rotate the first magnetic source about an axis parallel to the height of the first magnetic source.

In some embodiments, the method includes providing a third magnetic source positioned within the frame in proximity to the second magnetic source and applying the second magnetic field to the third magnetic source and causing the third magnetic source to rotate and produce a third magnetic field.

### BRIEF DESCRIPTION OF THE DRAWINGS

The accompanying drawings, which are included to provide a better understanding of the disclosure, illustrate embodiments of the disclosure and together with the description serve to explain the principle of the disclosure. In the drawings:

FIG. 1 illustrates a magnetic field therapy arrangement in accordance with aspects described herein;

FIGS. 2A-2D illustrate an example phase relationship between three diametrically magnetized magnets when rotating in accordance with aspects described herein;

FIG. 3 illustrates a magnetic field therapy system in accordance with aspects described herein;

FIG. 4 illustrates a magnetic field therapy arrangement in accordance with aspects described herein;

FIG. 5 illustrates a magnetic field therapy system in accordance with aspects described herein;

FIG. 6 illustrates a magnetic field therapy arrangement in accordance with aspects described herein;

FIG. 7 illustrates a magnetic field therapy system in accordance with aspects described herein;

FIG. 8 illustrates a magnetic field therapy system in accordance with aspects described herein;

FIG. 9 illustrates a magnetic field therapy arrangement in accordance with aspects described herein;

FIG. 10 illustrates a magnetic field therapy system in accordance with aspects described herein;

FIG. 11 illustrates a magnetic field therapy system in accordance with at least one embodiment described herein; in accordance with aspects described herein;

FIG. 12 illustrates a magnetic field therapy system in accordance with aspects described herein;

FIGS. 13A-B illustrate a magnetic field therapy arrangement in accordance with aspects described herein;

FIGS. 14A-B illustrate a magnetic field therapy arrangement in accordance with aspects described herein;

FIG. 15 is a flow diagram of a method for providing treatment to a subject in accordance with aspects described herein;

FIG. 16 is a flow diagram of a method for providing treatment to a subject in accordance with aspects described herein;

FIG. 17 is a flow diagram of a method for providing treatment to a subject in accordance with aspects described herein; and

FIG. 18 illustrates an example computing device.

### DETAILED DESCRIPTION

The disclosure relates to systems and methods that utilize remote drive techniques to provide magnetic field therapy.

As described above, magnetic field therapy systems are used to provide therapeutic treatment. Magnetic field pulses or waves stimulate nerve cells and neuronal circuitry in the brain to improve certain mental disorders such as schizophrenia, obsessive compulsive disorder (OCD), depression, and others. In some cases, alternating magnetic fields are generated by a moving magnet (or magnets) and administered to the subject. Magnetic field therapy is generally considered a “whole brain” treatment and the exact location of the magnet(s) over the subject's scalp can vary from treatment-to-treatment or subject-to-subject. In some cases, optimal stimulation is provided by a number of permanent magnets all moved rhythmically over the subject's scalp. Each permanent magnet is moved using a motor. Alternatively, the magnets are physically connected to each other with a shaft or belt, so that the magnets move synchronously, using only one motor. However, these components (e.g., motors, belts, etc.) can increase the weight, cost, and size of such therapy systems (or devices).

As such, remote drive techniques for providing magnetic field therapy are described herein. In at least one embodiment, a system for providing treatment includes a first magnetic source configured to produce a first magnetic field and a second magnetic source positioned in proximity to the first magnetic source. The first magnetic field is applied to the second magnetic source causing the second magnetic source to rotate and produce a second magnetic field. The first and second magnetic fields are applied to the head of the subject to provide a therapeutic treatment. In some examples, the first magnetic source is rotated by a motor to produce the first magnetic field. In some examples, the first magnetic source is a stationary magnetic source (e.g., an electromagnetic coil).

As discussed above, a magnet can be moved (or rotated) using a motor. Alternatively, a magnet can be moved (or rotated) using the influence of another magnetic field generator. In some examples, the other magnetic field generator is a permanent magnet or a coil (e.g., an electromagnetic coil). Magnets affect each other through the interaction of the magnetic fields generated by each magnet. For example, if two magnets are mounted with a shaft through the center so that each magnet is free to rotate, the magnets will naturally line up so that the ends with opposite magnetic poles are near to each other. In other words, the north pole of one magnet will be near the south pole of the other magnet. In some examples, a third magnet is mounted in-line with the first two magnets so that it also freely spins. The three magnets will naturally align so that opposite poles (e.g., north and south) are near to each other.

In a two-magnet configuration, if the first magnet is physically rotated, either using a motor or some other means, the resulting magnetic field will affect the second magnet. The magnetic field of the first magnet causes the second magnet to rotate as well, to maintain equilibrium. In some examples, if the magnets are bar-type, the rotation of the second magnet will be less smooth due to the non-uniform distance between the magnets during rotation. However, if the magnets are cylindrical and diametrically magnetized, the rotation of the second magnet will be smooth, rotating in phase with the first magnet. Likewise, if the magnets are cylindrical and diametrically magnetized, then a third magnet may be added in line with the first two magnets, and the third magnet will rotate in phase with the first two magnets.

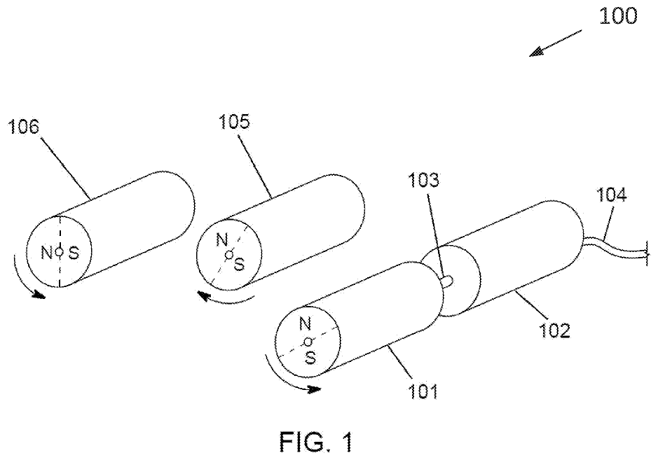

FIG. 1 illustrates a magnetic field therapy arrangement 100 in accordance with aspects described herein. In some examples, the magnetic field therapy arrangement 100 is configured to be incorporated in a therapy system or device that administers therapeutic treatments to a subject. As shown, the system 100 includes three rotating permanent magnets 101, 105, and 106. In some examples, each magnet is cylindrical and diametrically magnetized so that the north and south poles are located on the curved surface at opposite sides. In some examples, each magnet has a height and a diameter. The magnetization direction is through the diameter of each cylindrical magnet. In some examples, the first magnet 101 is rotated using a motor 102. In some examples, the motor 102 is coupled to the first magnet 101 via a shaft 103. In some examples, the motor is powered and controlled using at least one cable 104. While not shown, the at least one cable 104 is configured to be coupled to at least one controller or processor. The at least one controller is configured to operate the motor 102 and provide power to the motor 102. In some examples, the motor 103 receives power from a source other than the at least one controller (e.g., a battery).

In some examples, the motor 102 is configured to rotate the first magnet 101 in a first direction about an axis parallel to the height (e.g., long side) of the first magnet 101. In the illustrated example, the first magnet 101 is rotated in a counterclockwise direction, as shown by the curved arrow near the first magnet 101. When a diametrically magnetized cylindrical magnet is rotated (e.g., the first magnet 101), it generates a first rotating magnetic field, where the amplitude in any direction is approximately sinusoidal. The first rotating magnetic field generated by the first magnet 101 is applied to the second magnet 105, which is held stationary but allowed to freely rotate. In some examples, the second magnet 105 is positioned such that the height of the second magnet 105 is parallel to the height of the first magnet 101. In some examples, the second magnet 105 is held using a shaft and bearings (not shown). The magnetic field of the second magnet 105 opposes the first rotating magnetic field of the first magnet 101. As such, the first rotating magnetic field causes the second magnet 105 to rotate in a clockwise direction, as shown by the curved line near the second magnet 105. While rotating, the second magnet 105 generates a second rotating magnetic field that is applied to the third magnet 106, which is held stationary but allowed to freely rotate. In some examples, the third magnet 106 is positioned such that the height of the third magnet 106 is parallel to the height of the second magnet 105. In some examples, the third magnet 106 is held using a shaft and bearings (not shown). The magnetic field of the third magnet 106 opposes the second rotating magnetic field of the second magnet 105. As such, second rotating magnetic field causes the third magnet 106 to rotate in a counterclockwise direction, as shown by the curved line near the third magnet 106. It should be appreciated that the first magnet 101 may alternatively be rotated in a clockwise direction. In such examples, the second magnet 105 will rotate in a counterclockwise direction and the third magnet 106 will rotate in a clockwise direction.

In some examples, the motor 102 is configured to rotate the first magnet 101 at a first frequency. The rotation of the first magnet 101 at the first frequency causes the first rotating magnetic field of the first magnet 101 to have a second frequency. In some examples, the first rotating magnetic field of the first magnet 101 causes the second magnet 105 to rotate at a third frequency. The rotation of the second magnet 105 at the third frequency causes the second rotating magnetic field of the second magnet 105 to have a fourth frequency. In some examples, the second rotating magnetic field of the second magnet 105 causes the third magnet 106 to rotate at a fifth frequency. The rotation of the third magnet 106 at the fifth frequency causes the third rotating magnetic field of the third magnet 106 to have a sixth frequency. It should be appreciated that the rotational frequency of each magnet (e.g., cycles/see) is equal to the resulting magnetic field frequency (e.g., in Hz). In other words, the frequency of the rotating magnetic field corresponds to the speed (or frequency) at which the magnet rotates.

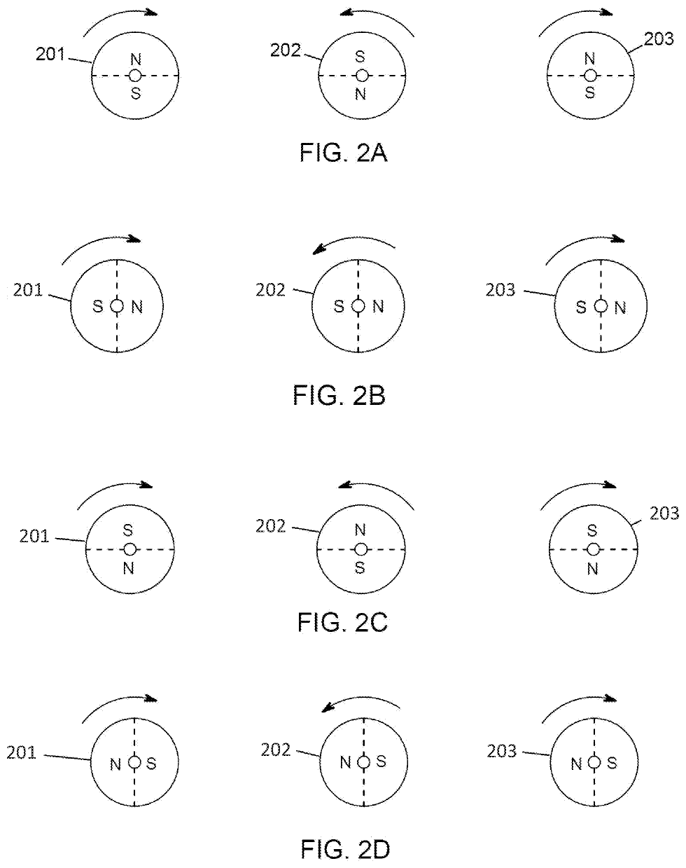

FIGS. 2A-2D illustrate the phase relationship between three diametrically magnetized magnets when rotating. As shown, all three magnets are in a line and approximately equally spaced. In the illustrated example, the first magnet 201 is rotated using a motor (or some other means). However, it should be appreciated that the relationship between the magnets is the same regardless of which of the three magnets is rotated.

FIG. 2A shows the rest position for the second magnet 202 and the third magnet 203 when the first magnet 201 is set to a position with the magnetic field axis perpendicular to a line between the magnets (e.g., through the center of each magnet). The direction arrows around the magnets 201-203 show the direction of rotation of each magnet (e.g., clockwise or counterclockwise). FIG. 2B shows the position of the second and third magnets 202, 203 when the first magnet 201 is oriented such that the magnetic field is in line with the line between the magnets. FIG. 2C and FIG. 2D continue to show the rotation sequence, with the second and third magnets 202, 203 oriented to oppose the magnetic field from the adjacent magnets.

The magnetic field generated by the first magnet 201 also affects the third magnet 203, however because the second magnet 202 is closer to the third magnet 203, the magnetic field of the second magnet 202 dominates. The lowest energy state of the three magnets 201-203 is shown in FIG. 2B and FIG. 2D, where the magnets line up NS-NS-NS or SN-SN-SN. To rotate the first magnet 201 from this position, a load is placed on the motor (e.g., the motor configured to rotate the first magnet 201). In some examples, this load increases if more magnets are added in the line. Eventually, either the motor will be unable to overcome the load, or the second magnet 202 will be unable to make a full rotation when the first magnet 201 is rotated, and will instead wobble back and forth as the first magnet 201 is rotated. Therefore, an upper limit on the number of magnets in a line is evident. In some examples, if the second magnet 202 is motorized instead of the first magnet 201, the load on the motor will not change appreciably, but the load on the first and third magnets 201, 203 will be reduced.

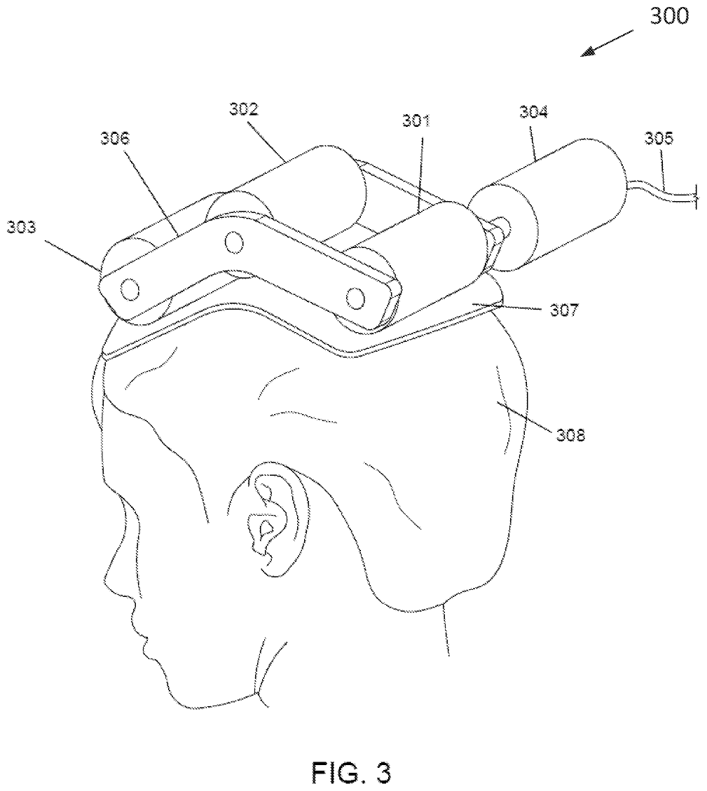

FIG. 3 illustrates a magnetic field therapy system 300 in accordance with aspects described herein. As shown, the system 300 includes three rotating permanent magnets 301, 302, and 303. In some examples, each magnet is cylindrical and diametrically magnetized so that the north and south poles are located on the curved surface at opposite sides. The magnetization direction is through the diameter of each cylindrical magnet. In some examples, the first magnet 301 is rotated using a motor 304. In some examples, the motor 304 is coupled to the first magnet 301 via a shaft. In some examples, the motor 304 is powered and controlled using at least one cable 305. While not shown, the at least one cable 305 is configured to be coupled to at least one controller or processor. The at least one controller is configured to operate the motor 304 and provide power to the motor 304. In some examples, the motor 304 receives power from a source other than the at least one controller (e.g., a battery).

The first magnet 301 generates an alternating magnetic field, which is applied to the second magnet 302 and causes the second magnet 302 to rotate. The magnetic field generated by the second magnet 302 is applied to the third magnet 303 and causes the third magnet 303 to rotate. In some examples, the three magnets 301-303 are housed in a frame 306 using shafts and bearings that allow them to rotate freely (e.g., with respect to the frame 306). In some examples, each shaft runs through the magnet parallel to the height (e.g., long side) of the magnet. In some examples, each shaft is coupled to the magnet such that rotation of the shaft causes the magnet to rotate. In some examples, the frame 306 is curved to conform to the shape of the head 308 of the subject. In some examples, a cover 307 is used to prevent the subject's hair from being caught in the shafts (or bearings) of the rotating magnets 301-303. In some examples, the cover 307 encircles the magnets 301-303. In some examples, the cover 307 encircles the magnets 301-303 and the motor 304 to protect the magnets and prevent injury to the subject.

As discussed above, magnetic field therapy is generally considered a “whole brain” treatment and the exact location of the magnet(s) over the subject's scalp can vary from treatment-to-treatment or subject-to-subject. In some examples, the frame 307 is adjustable to conform to different head sizes and shapes. In some examples, the frame 307 is configured such that the rotating magnets 301-303 are positioned as close as possible to the subject's scalp.

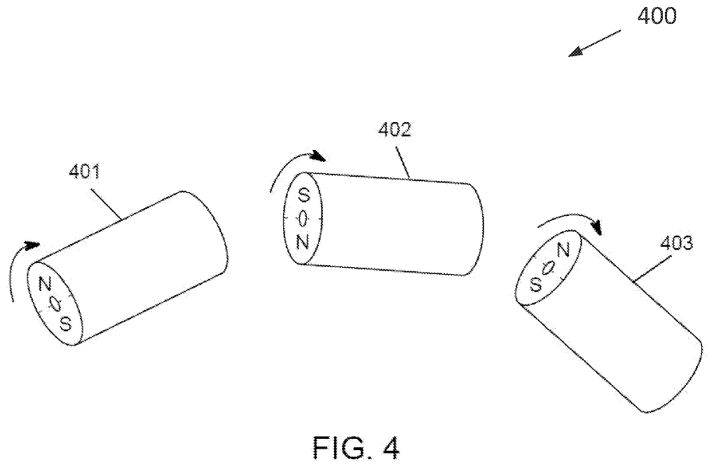

FIG. 4 illustrates a magnetic field therapy arrangement 400 in accordance with aspects described herein. In some examples, the magnetic field therapy arrangement 400 is configured to be incorporated in a therapy system or device that administers therapeutic treatments to a subject. As shown, the system 400 includes three rotating permanent magnets 401, 402, and 403. In some examples, each magnet is cylindrical and diametrically magnetized. In some examples, the first magnet 401 is rotated using a motor (not shown). The magnets 401-403 are positioned so that they are end-to-end (e.g., short side to short side). In some examples, the second magnet 402 is positioned such that a first end of the second magnet 402 faces an end of the first magnet 401 and the third magnet 403 is positioned such that an end of the third magnet 403 faces a second end of the second magnet 402. Given that the north and south poles attract, when the first magnet 401 is rotated, as shown by the arrow in the figure, the second magnet 402 will also rotate. The second magnet 402 rotates in the same direction as the first magnet 401, keeping the north and south poles as close as possible. The third magnet 403 is affected by the magnetic field from the second magnet 402 and rotates in the same direction.

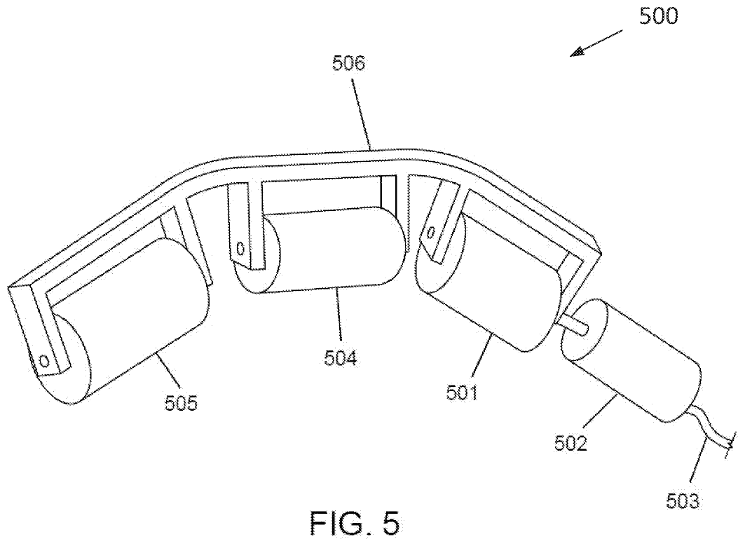

FIG. 5 illustrates a magnetic field therapy system 500 in accordance with aspects described herein. The system 500 includes three rotating permanent magnets 501, 504, and 505 mounted end-to-end in a frame 506. In some examples, the first magnet 501 is rotated using a motor 502. In some examples, the motor 502 is coupled to the first magnet 501 via a shaft. In some examples, the motor 502 is powered and controlled using at least one cable 503. While not shown, the at least one cable 503 is configured to be coupled to at least one controller. The at least one controller is configured to operate the motor 502 and provide power to the motor 502. In some examples, the motor 502 receives power from a source other than the at least one controller (e.g., a battery). The first magnet 501 rotates, causing the second magnet 504 and third magnet 505 to rotate in phase with the first magnet 501. The three magnets 501, 504, 505 are held in the frame 506 and configured to rotate freely with respect to the frame 506. In some examples, the frame 506 is curved so that the magnets can be positioned as close as possible to the subject's scalp when positioned over their head.

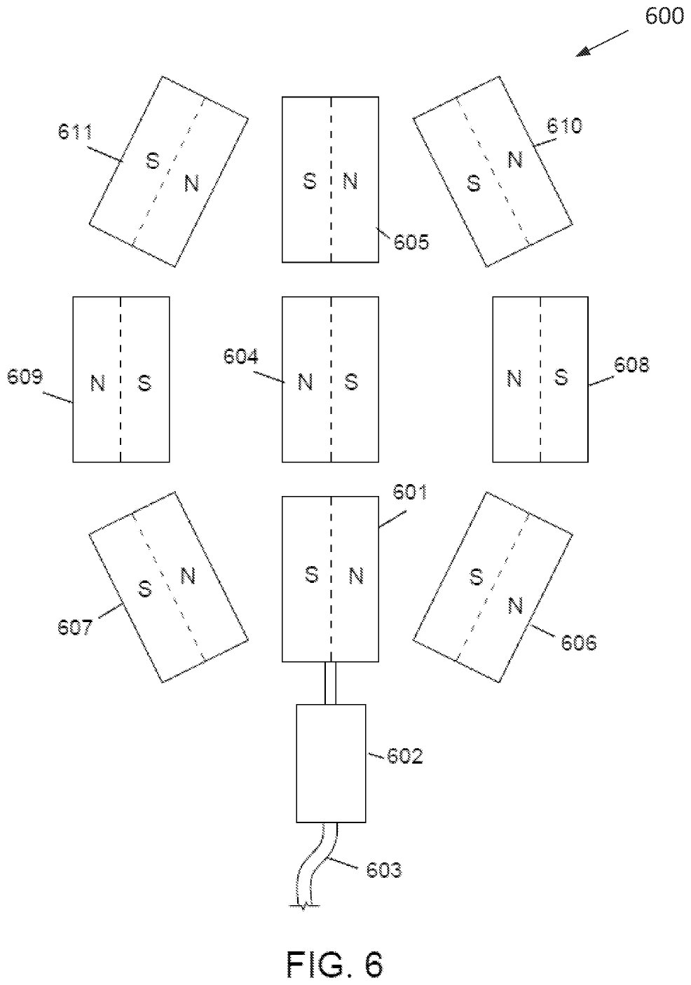

FIG. 6 illustrates a magnetic field therapy arrangement 600 in accordance with aspects described herein. In some examples, the magnetic field therapy arrangement 600 is configured to be incorporated in a therapy system or device that administers therapeutic treatments to a subject. As shown, several magnets are rotated in sync, with only one of the magnets being rotated through a motor. In the illustrated example, the first central magnet 601 is rotated using a motor 602 connected by a shaft. The motor is powered and controlled by a cable 603 to a driver (not shown). The magnetic field from the first magnet 601 results in the rotation of the three magnets closest to it (e.g., magnets 604, 606, 607). These magnets in turn, rotate the magnets closest to them (e.g., magnets 605, 608, 609), which also rotate the last set of magnets (e.g., magnets 610, 611). The magnetic field from each magnet combine to create a widely distributed therapeutic magnetic field.

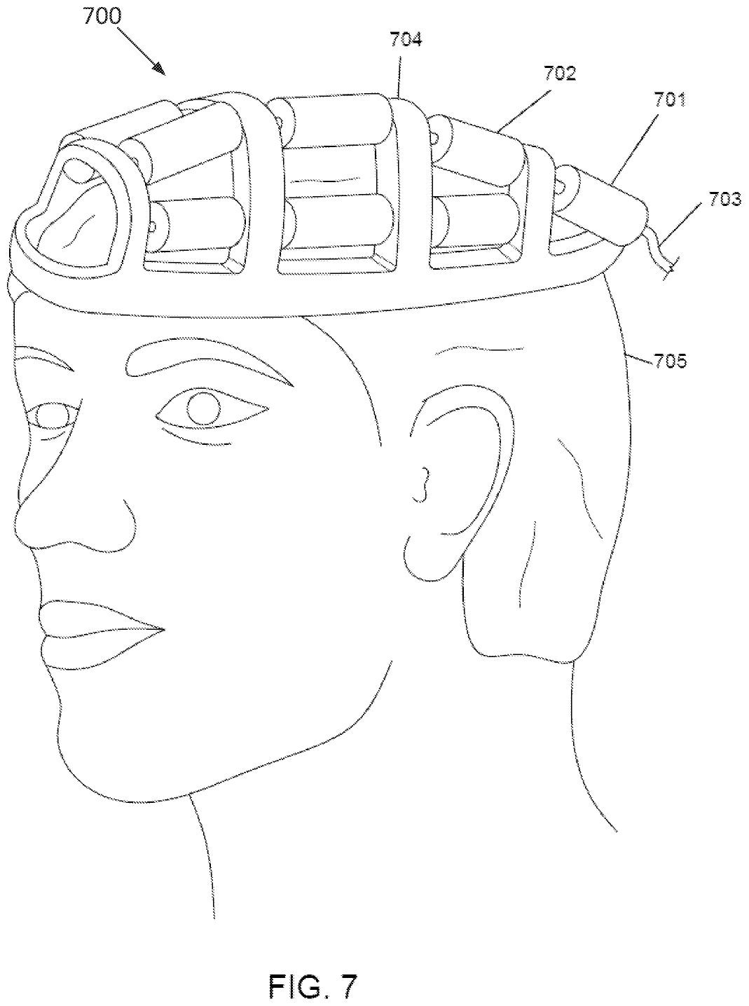

FIG. 7 illustrates a magnetic field therapy system 700 in accordance with aspects described herein. In some examples, the system 700 corresponds to the arrangement 600 of FIG. 6 incorporated into a helmet 704. The first magnet 702 is rotated through a shaft by a motor 701. The motor is powered and controlled by a driver (not shown) through a cable 703. In some examples, the driver is incorporated into the helmet 704. In some examples, the driver is external to the helmet 704. The helmet 704 is configured to keep the magnets as close as possible to the scalp of the subject 705. In some examples, the helmet 704 is designed to conform to the shape of the subject's head. In some examples, the helmet 704 includes an adjustable frame to account for different head sizes and shapes. In some examples, a cover (not shown) is incorporated into the helmet 704 to keep the subject's hair from winding onto the magnet shafts, and to protect the system 700.

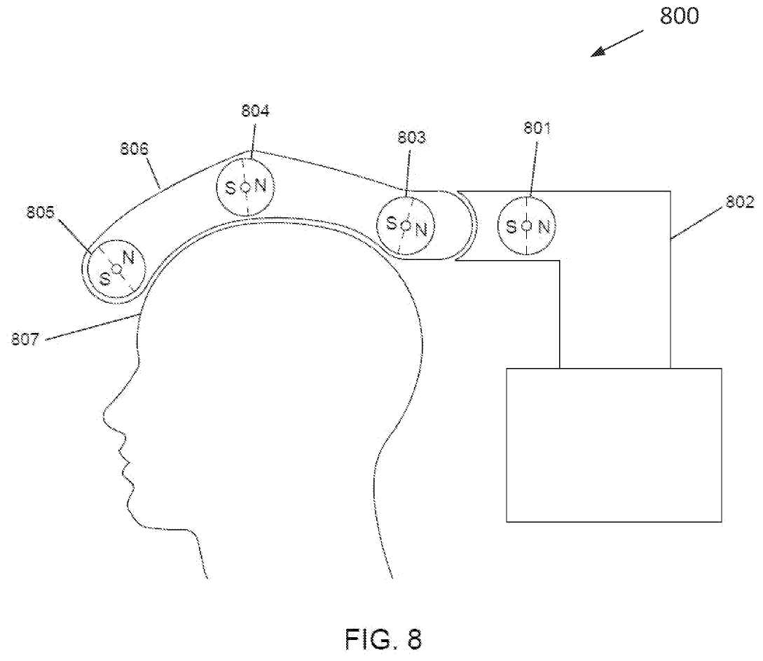

FIG. 8 illustrates a magnetic field therapy system 800 in accordance with aspects described herein. As shown, a magnet external to a helmet rotates magnets inside the helmet through the influence of its magnetic field. The drive magnet 801 is contained in a base station 802 and is rotated using a motor (not shown). When the helmet 806 is positioned next to the base station 802, the drive magnet 801 attracts the first magnet 803, holding the helmet 806 in place (e.g., adjacent to the base station 802). In some examples, a subject 807 wears the helmet 806 and sits or stands with their back to the base station 802. In some examples, the subject 807 lies down while wearing the helmet 806. When the drive magnet 801 is rotated, the drive magnet 801 produces a magnetic field that is applied to the first magnet 803 and causes the first magnet 803 to also rotate. The magnetic field from the first magnet 803 is applied to the second magnet 804 and causes the second magnet 804 to rotate. The magnetic field from the second magnet 804 is applied to the third magnet 805 and causes the third magnet 805 to rotate as well. If the subject 807 moves their head forward, disengaging the helmet 806 from the base station 802, then the first, second, and third magnets 803-805 in the helmet 806 will stop rotating, and magnetic therapy will end. In some examples, since the motor is included in the base station 802 rather than the helmet 806, the helmet 806 is lighter, more comfortable, and/or less expensive. In some examples, this configuration also removes the need for a cable from the helmet 806 to a motor driver or power source.

As described above, one or more permanent magnets can be moved using the influence of a magnetic field generated by another permanent magnet. In some examples, one or more permanent magnets are moved using the influence of a magnetic field generated by a magnetic coil (e.g., an electromagnet). The magnetic field generated by an electric current flowing through a coil of wire has a polarity and an intensity, and by altering these parameters, it is possible to move (or rotate) a permanent magnet. In some examples, the magnetic field intensity used to rotate a permanent magnet is less than the magnetic field of the permanent magnet itself. As such, one or more coils of wire can efficiently rotate a high strength permanent magnet, such as a Neodymium magnet, using only a fraction of the magnetic field strength of the permanent magnet.

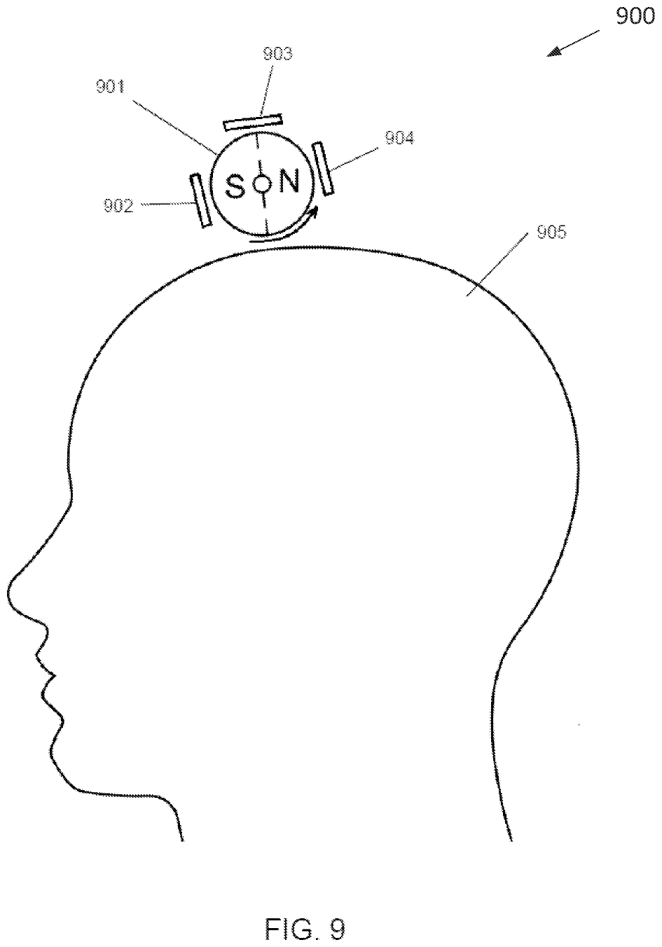

FIG. 9 illustrates a magnetic field therapy arrangement 900 in accordance with aspects described herein. In some examples, the magnetic field therapy arrangement 900 is configured to be incorporated in a therapy system or device that administers therapeutic treatments to a subject. In some examples, a single cylindrical diametrically magnetized permanent magnet 901 is configured to rotate above the head of a subject 905 to provide magnetic field therapy. Three electromagnetic coils 902, 903, 904 are distributed near the magnet 901 such that the axis of each coil is perpendicular to the surface of the magnet 901. The axis of each coil is an axis that extends through the center of the coil and is perpendicular to the coil itself. By varying the electric current amplitude and polarity through each coil 902-904, a magnetic field is created around the permanent magnet. The magnetic field is applied to the permanent magnet 901 and causes the permanent magnet 901 to rotate. In some examples, the amplitudes and/or polarities of the magnetic fields generated by the coils 902-904 are varied to push or pull the magnetic poles of the permanent magnet 901. In some examples, the magnetic field generated by the three coils 902-904 is very small compared to the magnetic field generated by the permanent magnet 901. Although the coils 902-904 generate a magnetic field, this magnetic field is not the primary therapeutic magnetic field. Instead, the high amplitude alternating magnetic field generated by the permanent magnet 901 is the primary therapeutic magnetic field administered to the subject 905.

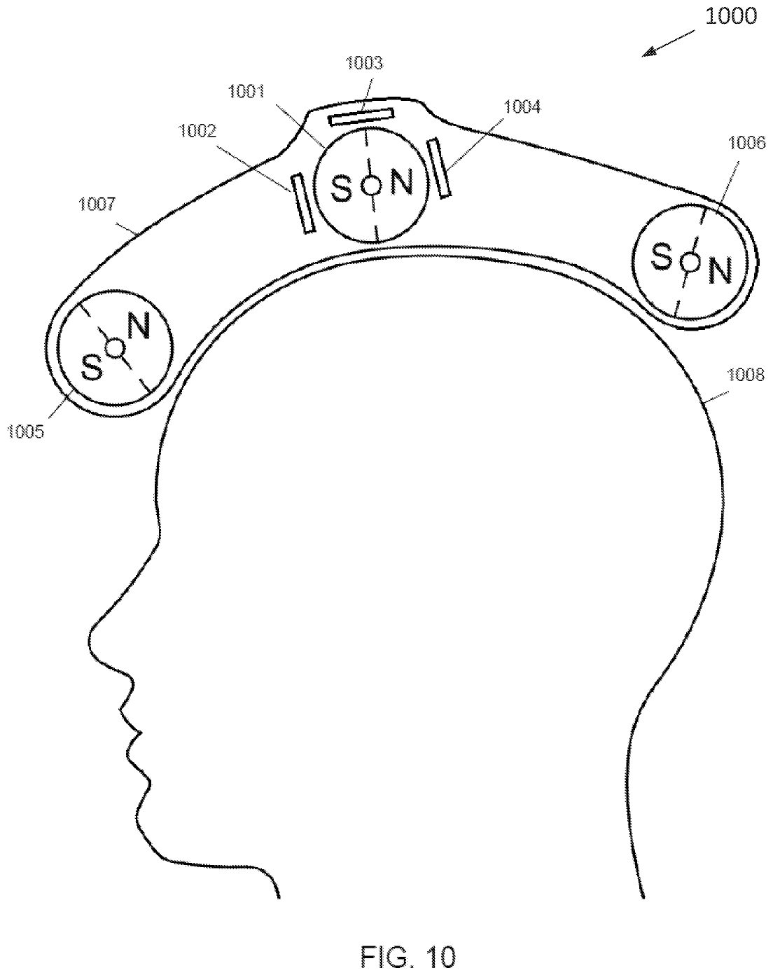

In some examples, the rotating permanent magnet (e.g., magnet 901) influences and causes nearby permanent magnets to rotate as well. FIG. 10 illustrates a magnetic field therapy system 1000 in accordance with aspects described herein. A shown, the system 1000 includes three permanent magnets where only the center permanent magnet 1001 is actively rotated using three electromagnetic coils 1002, 1003, 1004. The magnetic field produced by the central magnet 1001 is applied to the front magnet 1005 and the rear magnet 1006, causing them to rotate. In some examples, the front coil 1002 influences the front magnet 1005, but to a lesser extent than the magnetic field of the center magnet 1001. Likewise, in some examples, the rear coil 1004 influences the rear magnet 1006, but to a lesser extent than the magnetic field of the center magnet 1001. In some examples, the coils and magnets are all contained in a helmet 1007. In some examples, the helmet 1007 is worn by a subject 1008 receiving magnetic therapy.

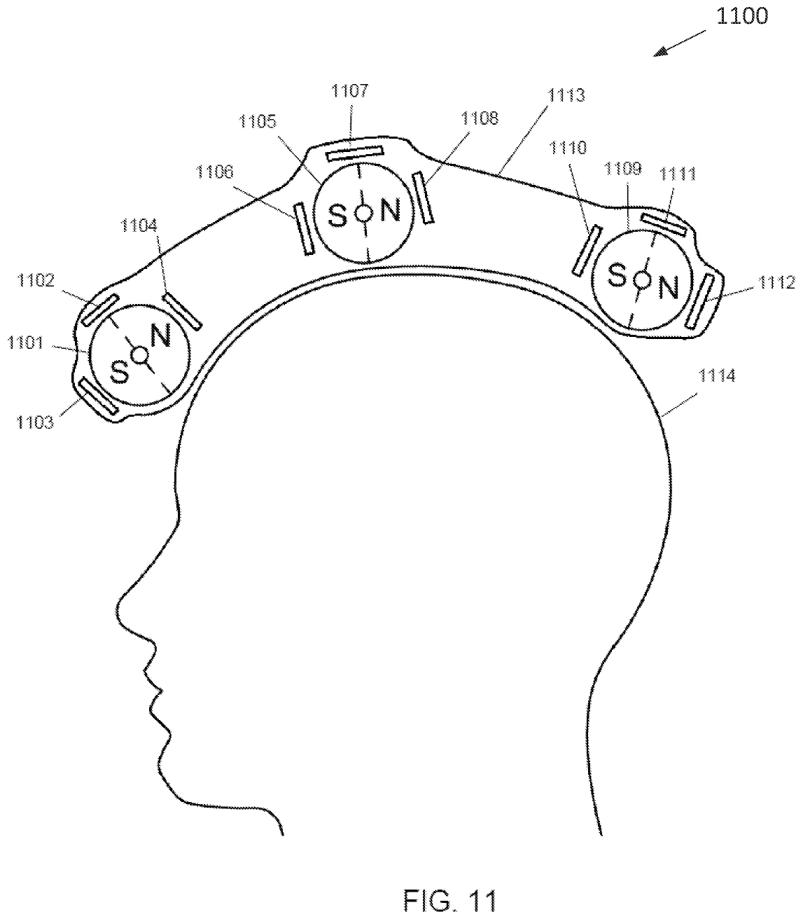

FIG. 11 illustrates a magnetic field therapy system 1100 in accordance with aspects described herein. In some examples, the system 1100 includes three cylindrical diametrically magnetized permanent magnets that are each rotated using coils. As shown, the front magnet 1101 is rotated using three electromagnetic coils 1102, 1103, 1104. The central magnet 1105 is rotated using the three electromagnetic coils 1106, 1107, 1108. The rear magnet 1109 is rotated using the three electromagnetic coils 1110, 1111, 1112. In some examples, the permanent magnets 1101, 1105, 1109 influence each other in manner which assists rotation. In some examples, the phase relationship between the three sets of coils is configured such that the permanent magnets 1101, 1105, and 1109 are moving in phase throughout rotation. In other words, if the magnetic fields generated by coils 1103, 1106, and 1110 are identical, the magnetic fields generated by coils 1102, 1107, and 1111 are identical, and the magnetic fields generated by coils 1104, 1108, and 1112 are identical, then the permanent magnets 1101, 1105, and 1109 will all rotate in phase with each other, because the coils will be pushing the poles of the magnets 1101, 1105, and 1109 in the same way. As such, the influence the permanent magnets 1101, 1105, and 1109 have on each other will assist rotation instead of hindering it. In some examples, the three magnets and coils are housed in a helmet 1113. In some examples, the helmet 1113 is worn by a subject 1114 to administer magnetic field therapy to the head of the subject 1114.

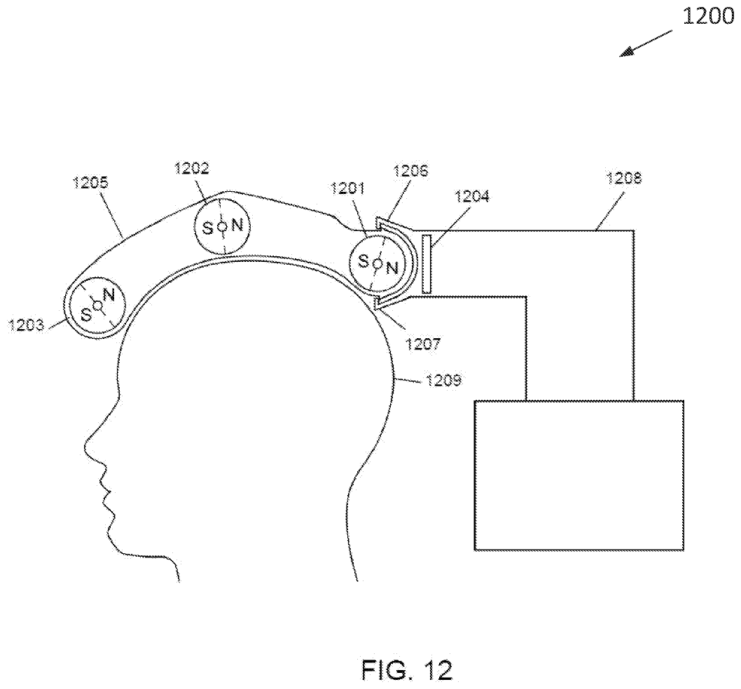

In some examples, permanent magnets are rotated using a coil that is not part of the helmet. In such examples, the coil is separate from the helmet but positioned in a location where it influences one or more of the rotating permanent magnets. FIG. 12 illustrates a magnetic field therapy system 1200 in accordance with aspects described herein. In some examples, the system 1200 includes a rear cylindrical diametrically magnetized permanent magnet 1201 that is influenced by a magnetic field generated from a nearby electromagnetic coil 1204. In some examples, the coil 1204 is held in a separate housing 1208 (e.g., a base station). The coil 1204 alternates the polarity of the generated magnetic field to attract either the north or south pole of the magnet alternately. By continuously alternating the polarity at a precise frequency, the magnetic field of the coil 1204 causes the rear magnet 1201 to rotate at a desired frequency. The rear magnet 1201 influences the central magnet 1202, causing it to rotate as well. The central magnet 1202 influences the front magnet 1203, causing it to rotate. In some examples, all three magnets 1201-1203 are housed in a helmet 1205 and provide magnetic field therapy to the head of the subject 1209. In some examples, since the coil 1204 generates an alternating north-south field, it will tend to push and pull the helmet 1205. In some examples, the helmet 1205 is held in place, so that the push/pull force can be used to rotate the rear magnet 1201 instead of moving the entire helmet 1205. In some examples, the helmet 1205 is held in place using two clamps 1206, 1207. In such examples, the subject 1209 puts the helmet 1205 on and leans back into the housing 1208 so that the clamps 1206, 1207 hold the helmet 1205 in place. When therapy is complete, the subject 1209 pulls the helmet 1205 away from the housing 1208, forcing the clamps 1206, 1207 to release. In some examples, the helmet is held in place using a different means, such as screws, belts, Velcro, pins, or other mechanisms.

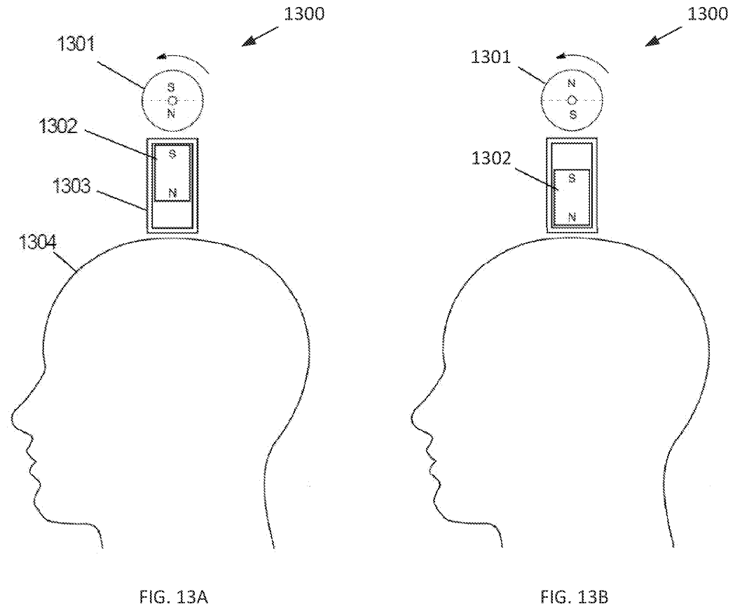

FIGS. 13A and 13B illustrate a magnetic field therapy arrangement 1300 in accordance with aspects described herein. In some examples, the magnetic field therapy arrangement 1300 is configured to be incorporated in a therapy system or device that administers therapeutic treatments to a subject. In some examples, a linear motion is imparted to a bar magnet 1302, which comprises a south and north pole at each end. In some examples, the bar magnet 1302 is held in an enclosure 1303, which is fixed in place relative to a head of a subject 1304. In some examples, the enclosure 1303 ensures that the bar magnet 1302 is at a fixed orientation relative to the scalp, and that the bar magnet 1302 is allowed to move perpendicularly to the scalp. A diametrically magnetized cylindrical permanent magnet 1301 is fixed in place near one end of the enclosure 1303 opposite to the head of the subject 1304. When the permanent magnet 1301 is rotated, a push-pull force is imparted on the bar magnet 1302 in the enclosure 1303. The imparted force causes the bar magnet 1302 to move back and forth between the ends of the enclosure 1303. In FIG. 13A, the bar magnet 1302 has the south pole closest to the permanent magnet 1301. When the magnet 1301 is rotated such that the north side is closest to the bar magnet 1302, the bar magnet 1302 is pulled away from the scalp. As shown in FIG. 13B, when the magnet 1301 is rotated farther such that the south side is closest to the bar magnet 1302, the bar magnet 1302 is pushed towards the scalp. In some examples, the linear movement of the bar magnet 1302 provides an approximately unipolar magnetic field that is applied and removed from the scalp. In some examples, the approximately unipolar magnetic field is applied as therapy for the subject 1304. In some examples, the enclosure 1303 includes two non-ferrous springs that are positioned on either end of the bar magnet 1302. In some examples, the springs serve to reduce or eliminate the impact of the bar magnet 1302 striking one end of the enclosure 1303. In some examples, the springs help to reduce the sound or vibration of the enclosure 1303.

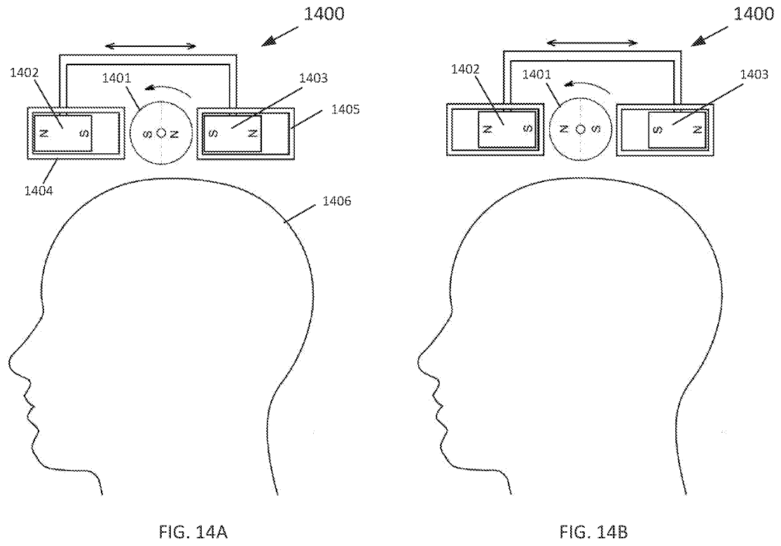

FIGS. 14A and 14B illustrate a magnetic field therapy arrangement 1400 in accordance with aspects described herein. In some examples, the magnetic field therapy arrangement 1400 is configured to be incorporated in a therapy system or device that administers therapeutic treatments to a subject. In some examples, a rotational motion is imparted on a diametrically magnetized cylindrical permanent magnet 1401 using two bar magnets 1402 and 1403. In some examples, the bar magnet 1402 is held in an enclosure 1404 and the bar magnet 1403 is held in an enclosure 1405. Both enclosures 1404, 1405 are fixed in place relative to a head of a subject 1406. In some examples, the enclosures 1404, 1405 ensure that the bar magnets 1402, 1403 are at a fixed orientation relative to the scalp. The bar magnets 1402, 1403 are moved in a linear motion which causes the permanent magnet 1401 to rotate. In some examples, the bar magnets 1402, 1403 are moved in unison. In some examples, the bar magnets 1402, 1403 are moved separately. In some examples, the bar magnets 1402, 1403 are moved via one or more actuators (not shown). In FIG. 14A, the south pole of the bar magnet 1403 is closest to the permanent magnet 1401. When the magnet 1401 is rotated such that the north side is closest to the bar magnet 1403, the magnet 1401 is rotated such that the north side of the magnet 1401 rotates away from the bar magnet 1403. Likewise, as shown in FIG. 14B, when the south pole of the bar magnet 1402 is closes to north side of the magnet 1401, the magnet 1401 is rotated such that the north side of the magnet 1401 rotates away from the bar magnet 1402. In some examples, the rotational movement of the magnet 1401 provides a magnetic field that is applied to the scalp. In some examples, the magnetic field is applied as therapy for the subject 1406.

As described above, the various magnetic therapy systems and arrangements provided herein are configured to provide treatment (e.g., brain stimulation) to the subject's brain. In some examples, the treatment device is configured to provide magnetic brain stimulation (e.g., transcranial magnetic stimulation (TMS) or repetitive TMS). In some examples, the treatment corresponds to a treatment plan (or treatment settings). In some examples, the treatment is directed to improving the symptoms of Autism Spectrum Disorder, Alzheimer's disease, ADHD, schizophrenia, anxiety, depression, coma, Parkinson's disease, substance abuse, bipolar disorder, sleep disorder, eating disorder, tinnitus, traumatic brain injury, post-traumatic stress disorder, or fibromyalgia. In some examples, the treatment device is configured to be worn on the subject's head while receiving treatment. In some examples, the treatment device provides sensory stimulation including flashing light, sound, video, or touch.

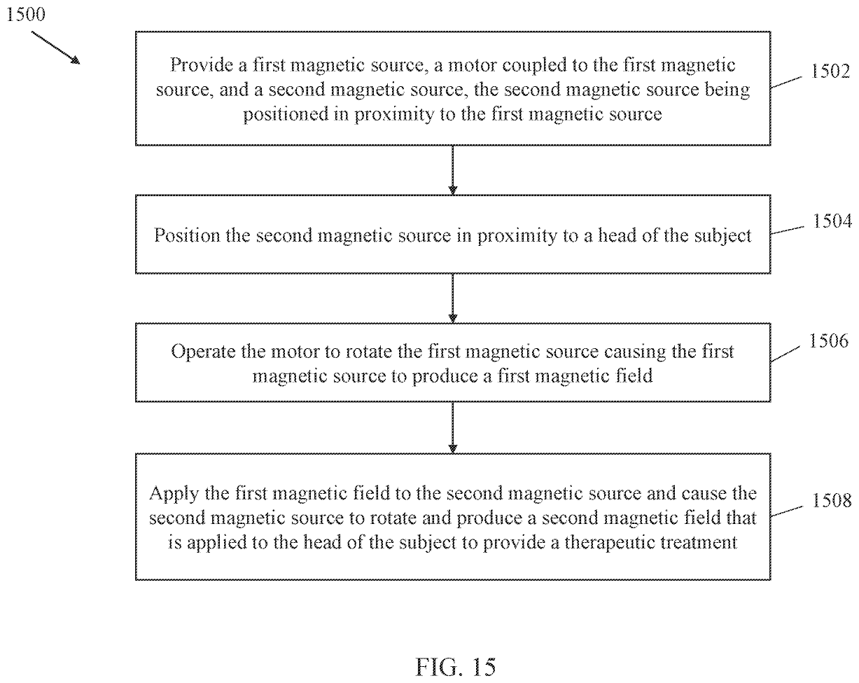

FIG. 15 is a flow diagram of a method 1500 for providing treatment to a subject in accordance with aspects described herein. In some examples, the method 1500 is configured to be carried out using the magnetic field therapy arrangement 100 of FIG. 1. It should be appreciated that the method 1500 may be carried out using different systems/arrangements.

At block 1502, a first magnetic source (e.g., first magnet 101), a motor (e.g., motor 102) coupled to the first magnetic source, and a second magnetic source (e.g., second magnet 105) are provided. The second magnetic source is positioned in proximity to the first magnetic source.

At block 1504, the second magnetic source is positioned in proximity to a head of the subject.

At block 1506, the motor is operated to rotate the first magnetic source causing the first magnetic source to produce a first magnetic field.

At block 1508, the first magnetic field is applied to the second magnetic source and causes the second magnetic source to rotate and produce a second magnetic field that is applied to the head of the subject to provide a therapeutic treatment. In some examples, the therapeutic treatment includes TMS.

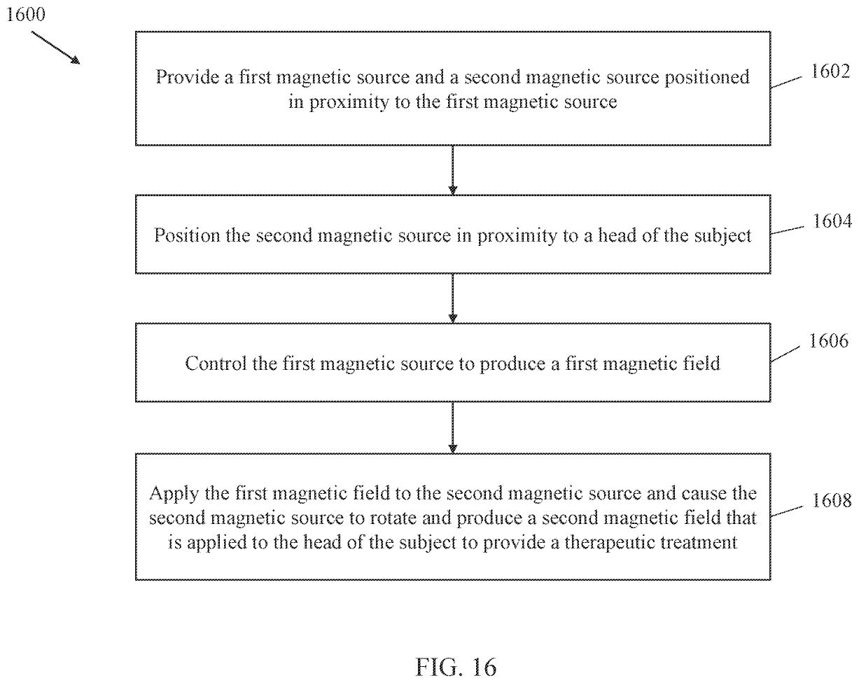

FIG. 16 is a flow diagram of a method 1600 for providing treatment to a subject in accordance with aspects described herein. In some examples, the method 1600 is configured to be carried out using the magnetic field therapy arrangement 900 of FIG. 9. It should be appreciated that the method 1600 may be carried out using different systems/arrangements.

At block 1602, a first magnetic source (e.g., electromagnetic coil(s) 902, 903, or 904) and a second magnetic source (e.g., magnet 901) are provided. The second magnetic source is positioned in proximity to the first magnetic source.

At block 1604, the second magnetic source is positioned in proximity to a head of the subject.

At block 1606, the first magnetic source is controlled to produce a first magnetic field. In some examples, controlling the first magnetic source to produce the first magnetic field includes providing a current to the first magnetic source to produce the first magnetic field.

At block 1608, the first magnetic field is applied to the second magnetic source and causes the second magnetic source to rotate and produce a second magnetic field that is applied to the head of the subject to provide a therapeutic treatment. In some examples, the therapeutic treatment includes TMS.

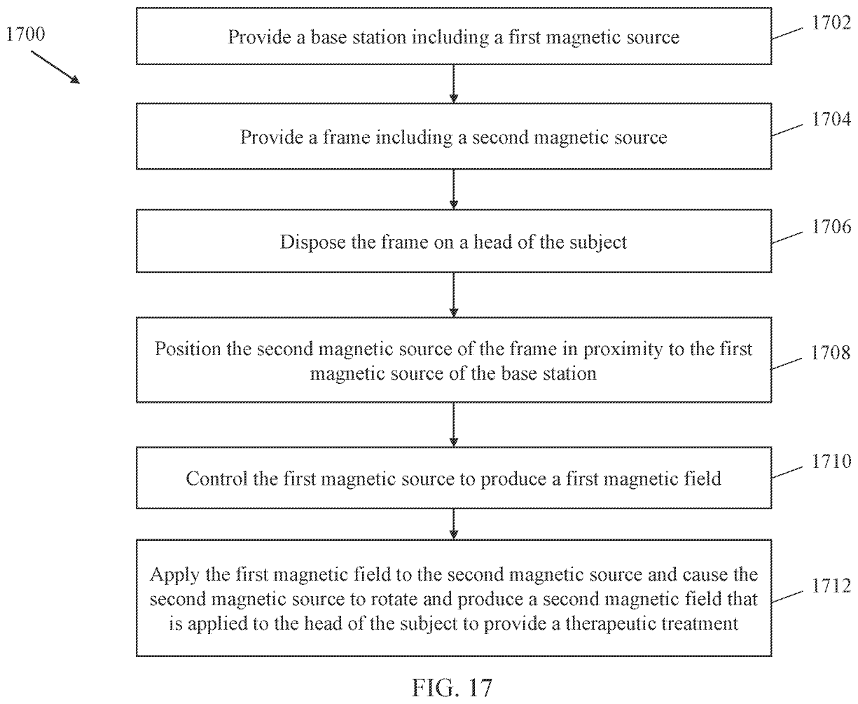

FIG. 17 is a flow diagram of a method 1700 for providing treatment to a subject in accordance with aspects described herein. In some examples, the method 1700 is configured to be carried out using the magnetic field therapy system 800 of FIG. 8 or the magnetic field therapy system 1200 of FIG. 12. It should be appreciated that the method 1700 may be carried out using different systems/arrangements.

At block 1702, a base station (e.g., base station 802) is provided including a first magnetic source (e.g., drive magnet 801).

At block 1704, a frame (e.g., helmet 806) is provided including a second magnetic source (e.g., magnet(s) 803, 804, or 805).

At block 1706, the frame is disposed on a head of the subject.

At block 1708, the second magnetic source of the frame is positioned in proximity to the first magnetic source of the base station. In some examples, the frame is positioned to be adjacent to the base station.

At block 1710, the first magnetic source is controlled to produce a first magnetic field. In some examples, controlling the first magnetic source to produce the first magnetic field includes providing a current to the first magnetic to produce the first magnetic field. In some examples, controlling the first magnetic source to produce the first magnetic field includes operating a motor (e.g., in the base station) to rotate the first magnetic source.

At block 1712, the first magnetic field is applied to the second magnetic source and causes the second magnetic source to rotate and produce a second magnetic field that is applied to the head of the subject to provide a therapeutic treatment. In some examples, the therapeutic treatment includes TMS.

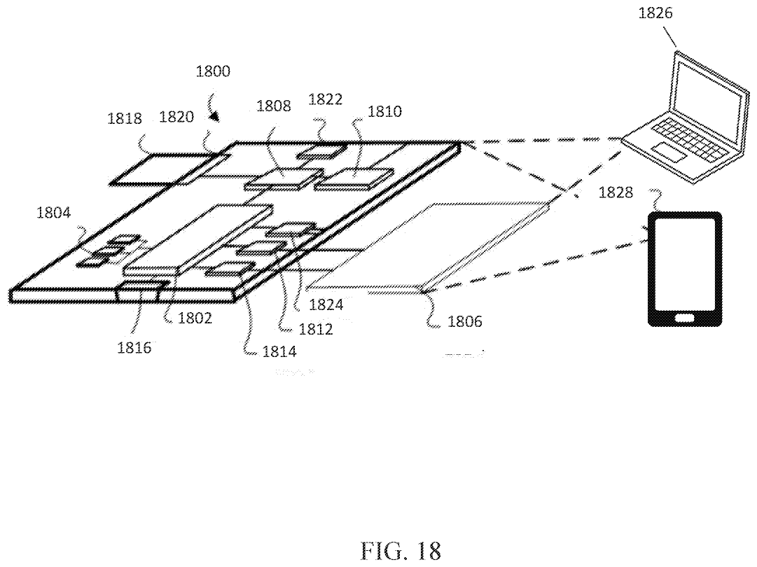

FIG. 18 shows an example of a generic computing device 1800, which may be used with some of the techniques described in this disclosure (e.g., to control or operate the magnetic field therapy systems and arrangements described herein). Computing device 1800 includes a processor 1802, memory 1804, an input/output device such as a display 1806, a communication interface 1808, and a transceiver 1810, among other components. The device 1800 may also be provided with a storage device, such as a micro-drive or other device, to provide additional storage. Each of the components 1800, 1802, 1804, 1806, 1808, and 1810, are interconnected using various buses, and several of the components may be mounted on a common motherboard or in other manners as appropriate.

The processor 1802 can execute instructions within the computing device 1800, including instructions stored in the memory 1804. The processor 1802 may be implemented as a chipset of chips that include separate and multiple analog and digital processors. The processor 1802 may provide, for example, for coordination of the other components of the device 1800, such as control of user interfaces, applications run by device 1800, and wireless communication by device 1800.

Processor 1802 may communicate with a user through control interface 1812 and display interface 1814 coupled to a display 1806. The display 1806 may be, for example, a TFT LCD (Thin-Film-Transistor Liquid Crystal Display) or an OLED (Organic Light Emitting Diode) display, or other appropriate display technology. The display interface 1814 may comprise appropriate circuitry for driving the display 1806 to present graphical and other information to a user. The control interface 1812 may receive commands from a user and convert them for submission to the processor 1802. In addition, an external interface 1816 may be provided in communication with processor 1802, so as to enable near area communication of device 1800 with other devices. External interface 1816 may provide, for example, for wired communication in some implementations, or for wireless communication in other implementations, and multiple interfaces may also be used.

The memory 1804 stores information within the computing device 1800. The memory 1804 can be implemented as one or more of a computer-readable medium or media, a volatile memory unit or units, or a non-volatile memory unit or units. Expansion memory 1818 may also be provided and connected to device 1800 through expansion interface 1820, which may include, for example, a SIMM (Single In Line Memory Module) card interface. Such expansion memory 1818 may provide extra storage space for device 1800, or may also store applications or other information for device 1800. Specifically, expansion memory 1818 may include instructions to carry out or supplement the processes described above, and may include secure information also. Thus, for example, expansion memory 1818 may be provided as a security module for device 1800, and may be programmed with instructions that permit secure use of device 1800. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 1804, expansion memory 1818, memory on processor 1802, or a propagated signal that may be received, for example, over transceiver 1810 or external interface 1816.

Device 1800 may communicate wirelessly through communication interface 1808, which may include digital signal processing circuitry where necessary. Communication interface 1808 may in some cases be a cellular modem. Communication interface 1808 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1810. In addition, short-range communication may occur, such as using a Bluetooth, WiFi, or other such transceiver (not shown). In addition, GPS (Global Positioning System) receiver module 1822 may provide additional navigation—and location-related wireless data to device 1800, which may be used as appropriate by applications running on device 1800.

Device 1800 may also communicate audibly using audio codec 1824, which may receive spoken information from a user and convert it to usable digital information. Audio codec 1824 may likewise generate audible sound for a user, such as through a speaker, e.g., in a handset of device 1800. Such sound may include sound from voice telephone calls, may include recorded sound (e.g., voice messages, music files, etc.) and may also include sound generated by applications operating on device 1800. In some examples, the device 1800 includes a microphone to collect audio (e.g., speech) from a user. Likewise, the device 1800 may include an input to receive a connection from an external microphone.

The computing device 1800 may be implemented in a number of different forms, as shown in FIG. 18. For example, it may be implemented as a computer (e.g., laptop) 1826. It may also be implemented as part of a smartphone 1828, smart watch, tablet, personal digital assistant, or other similar mobile device.

Some implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language resource), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending resources to and receiving resources from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

It will be appreciated to those skilled in the art that the preceding examples and embodiments are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure. It shall also be noted that elements of any claims may be arranged differently including having multiple dependencies, configurations, and combinations.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A system for providing treatment to a subject, comprising:
a first magnetic source;
a motor coupled to the first magnetic source;
a second magnetic source positioned in proximity to the first magnetic source;
at least one memory storing computer-executable instructions; and
at least one processor for executing the instructions stored on the memory, wherein execution of the instructions causes the at least one processor to:
when the second magnetic source is positioned in proximity to a head of the subject, operate the motor to rotate the first magnetic source causing the first magnetic source to produce a first magnetic field,
wherein the first magnetic field is applied to the second magnetic source and causes the second magnetic source to rotate and produce a second magnetic field that is applied to the head of the subject to provide a therapeutic treatment.

2. The system of claim 1, wherein the therapeutic treatment includes transcranial magnetic stimulation (TMS).

3. The system of claim 1, wherein the first magnetic field and the second magnetic field are applied to the head of the subject to provide the therapeutic treatment.

4. The system of claim 1, wherein the first magnetic source and the second magnetic source are permanent magnets.

5. The system of claim 1, wherein the first magnetic source and the second magnetic source are each cylindrical magnets having a height and a diameter.

6. The system of claim 5, wherein the motor is configured to rotate the first magnetic source in a first direction about an axis parallel to the height of the first magnetic source.

7. The system of claim 6, wherein the second magnetic source is positioned such that the first magnetic field causes the second magnetic source to rotate in a second direction, the second direction being opposite from the first direction.

8. The system of claim 7, wherein the height of the second magnetic source is parallel to the height of the first magnetic source.

9. The system of claim 6, wherein the second magnetic source is positioned such that the first magnetic field causes the second magnetic source to rotate in the first direction.

10. The system of claim 9, wherein an end of the second magnetic source faces an end of the first magnetic source, the end of the second magnetic source corresponding to a surface having the diameter of the second magnetic source and the end of the first magnetic source corresponding to a surface having the diameter of the first magnetic source.

11. The system of claim 6, further comprising:
a third magnetic source positioned in proximity to the second magnetic source, wherein the second magnetic field is applied to the third magnetic source and causes the third magnetic source to rotate and produce a third magnetic field.

12. The system of claim 11, wherein the third magnetic source is positioned such that the second magnetic field causes the third magnetic source to rotate in the first direction.

13. The system of claim 12, wherein the third magnetic source is a cylindrical magnet having a height and a diameter.

14. The system of claim 13, wherein the height of the third magnetic source is parallel to the height of the second magnetic source.

15. The system of claim 13, wherein an end of the third magnetic source faces an end of the second magnetic source, the end of the third magnetic source corresponding to a surface having the diameter of the third magnetic source and the end of the second magnetic source corresponding to a surface having the diameter of the second magnetic source.

16. The system of claim 1, wherein the motor is configured to rotate the first magnetic source at a first frequency.

17. The system of claim 16, wherein the rotation of the first magnetic source at the first frequency causes the first magnetic field to have a second frequency.

18. The system of claim 17, wherein the first magnetic field, when applied to the second magnetic source, causes the second magnetic source to rotate at a third frequency.

19. The system of claim 18, wherein the rotation of the second magnetic source at the third frequency causes the second magnetic field to have a fourth frequency.

20. A method of treating a subject, comprising:
providing a first magnetic source, a motor coupled to the first magnetic source, and a second magnetic source, the second magnetic source being positioned in proximity to the first magnetic source;
positioning the second magnetic source in proximity to a head of the subject;
operating the motor to rotate the first magnetic source causing the first magnetic source to produce a first magnetic field; and
applying the first magnetic field to the second magnetic source and causing the second magnetic source to rotate and produce a second magnetic field that is applied to the head of the subject to provide a therapeutic treatment.

21. The method of claim 20, wherein the therapeutic treatment includes transcranial magnetic stimulation (TMS).

22. The method of claim 20, further comprising:
positioning the first magnetic source in proximity to the head of the subject,
wherein the first magnetic field and the second magnetic field are applied to the head of the subject to provide the therapeutic treatment.

23. The method of claim 20, wherein the first magnetic source and the second magnetic source are permanent magnets.

24. The method of claim 20, wherein the first magnetic source and the second magnetic source are each cylindrical magnets having a height and a diameter.

25. The method of claim 24, wherein operating the motor to rotate the first magnetic source includes rotating the first magnetic source in a first direction about an axis parallel to the height of the first magnetic source.

26. The method of claim 25, wherein the second magnetic source is positioned such that the first magnetic field causes the second magnetic source to rotate in a second direction, the second direction being opposite from the first direction.

27. The method of claim 26, wherein the height of the second magnetic source is parallel to the height of the first magnetic source.

28. The method of claim 25, wherein the second magnetic source is positioned such that the first magnetic field causes the second magnetic source to rotate in the first direction.

29. The method of claim 28, wherein an end of the second magnetic source faces an end of the first magnetic source, the end of the second magnetic source corresponding to a surface having the diameter of the second magnetic source and the end of the first magnetic source corresponding to a surface having the diameter of the first magnetic source.

30. The method of claim 25, further comprising:
providing a third magnetic source positioned in proximity to the second magnetic source; and
applying the second magnetic field to the third magnetic source and causing the third magnetic source to rotate and produce a third magnetic field.

31. The method of claim 30, wherein the third magnetic source is positioned such that the second magnetic field causes the third magnetic source to rotate in the first direction.

32. The method of claim 31, wherein the third magnetic source is a cylindrical magnet having a height and a diameter.

33. The method of claim 32, wherein the height of the third magnetic source is parallel to the height of the second magnetic source.

34. The method of claim 32, wherein an end of the third magnetic source faces an end of the second magnetic source, the end of the third magnetic source corresponding to a surface having the diameter of the third magnetic source and the end of the second magnetic source corresponding to a surface having the diameter of the second magnetic source.

35. The method of claim 20, wherein operating the motor to rotate the first magnetic source includes rotating the first magnetic source at a first frequency.

36. The method of claim 35, wherein the rotation of the first magnetic source at the first frequency causes the first magnetic field to have a second frequency.

37. The method of claim 36, wherein the first magnetic field, when applied to the second magnetic source, causes the second magnetic source to rotate at a third frequency.

38. The method of claim 37, wherein the rotation of the second magnetic source at the third frequency causes the second magnetic field to have a fourth frequency.

39. A system for providing treatment to a subject, comprising:
a first magnetic source;
a second magnetic source positioned in proximity to the first magnetic source;
at least one memory storing computer-executable instructions; and
at least one processor for executing the instructions stored on the memory, wherein execution of the instructions causes the at least one processor to:
when in proximity to a head of the subject, control the first magnetic source to produce a first magnetic field,
wherein the first magnetic field is applied to the second magnetic source and causes the second magnetic source to rotate and produce a second magnetic field that is applied to the head of the subject to provide a therapeutic treatment.

40. The system of claim 39, wherein the first magnetic source is a stationary magnetic source.

41. The system of claim 39, wherein the first magnetic source is an electromagnetic coil.

42. The system of claim 41, wherein controlling the first magnetic source to produce the first magnetic field includes providing a current to the first magnetic source to produce the first magnetic field.

43. The system of claim 39, wherein the therapeutic treatment includes transcranial magnetic stimulation (TMS).

44. The system of claim 39, wherein the first magnetic field and the second magnetic field are applied to the head of the subject to provide the therapeutic treatment.

45. The system of claim 39, wherein the second magnetic source is a permanent magnet.

46. The system of claim 39, wherein the second magnetic source is a cylindrical magnet having a height and a diameter.

47. The system of claim 46, wherein the second magnetic source is positioned such that the first magnetic field causes the second magnetic source to rotate in a first direction.

48. The system of claim 46, further comprising:
a third magnetic source positioned in proximity to the second magnetic source, wherein the second magnetic field is applied to the third magnetic source and causes the third magnetic source to rotate and produce a third magnetic field.

49. The system of claim 48, wherein the third magnetic source is positioned such that the second magnetic field causes the third magnetic source to rotate in a second direction, the second direction being opposite from the first direction.

50. The system of claim 49, wherein the third magnetic source is a cylindrical magnet having a height and a diameter.

51. The system of claim 50, wherein the height of the third magnetic source is parallel to the height of the second magnetic source.

52. The system of claim 50, wherein an end of the third magnetic source faces an end of the second magnetic source, the end of the third magnetic source corresponding to a surface having the diameter of the third magnetic source and the end of the second magnetic source corresponding to a surface having the diameter of the second magnetic source.

53. A method of treating a subject, comprising:
providing a first magnetic source and a second magnetic source positioned in proximity to the first magnetic source;
positioning the second magnetic source in proximity to a head of the subject;
controlling the first magnetic source to produce a first magnetic field; and
applying the first magnetic field to the second magnetic source and causing the second magnetic source to rotate and produce a second magnetic field that is applied to the head of the subject to provide a therapeutic treatment.

54. The method of claim 53, wherein the first magnetic source is a stationary magnetic source.

55. The method of claim 53, wherein the first magnetic source is an electromagnetic coil.

56. The method of claim 55, wherein controlling the first magnetic source to produce the first magnetic field includes providing a current to the first magnetic source to produce the first magnetic field.

57. The method of claim 53, wherein the therapeutic treatment includes transcranial magnetic stimulation (TMS).

58. The method of claim 53, further comprising:
positioning the first magnetic source in proximity to the head of the subject,
wherein the first magnetic field and the second magnetic field are applied to the head of the subject to provide the therapeutic treatment.

59. The method of claim 53, wherein the second magnetic source is a permanent magnet.

60. The method of claim 53, wherein the second magnetic source is a cylindrical magnet having a height and a diameter.

61. The method of claim 60, wherein the second magnetic source is positioned such that the first magnetic field causes the second magnetic source to rotate in a first direction.

62. The method of claim 60, further comprising:
providing a third magnetic source positioned in proximity to the second magnetic source; and
applying the second magnetic field to the third magnetic source and causing the third magnetic source to rotate and produce a third magnetic field.

63. The method of claim 62, wherein the third magnetic source is positioned such that the second magnetic field causes the third magnetic source to rotate in a second direction, the second direction being opposite from the first direction.

64. The method of claim 63, wherein the third magnetic source is a cylindrical magnet having a height and a diameter.

65. The method of claim 64, wherein the height of the third magnetic source is parallel to the height of the second magnetic source.

66. The method of claim 64, wherein an end of the third magnetic source faces an end of the second magnetic source, the end of the third magnetic source corresponding to a surface having the diameter of the third magnetic source and the end of the second magnetic source corresponding to a surface having the diameter of the second magnetic source.

67. A wearable device for providing treatment to a subject, comprising:
a frame configured to be disposed on a head of the subject;
a first magnetic source positioned within the frame;
a motor coupled to the first magnetic source;
a second magnetic source positioned within the frame and in proximity to the first magnetic source;
at least one memory storing computer-executable instructions; and
at least one processor for executing the instructions stored on the memory, wherein execution of the instructions causes the at least one processor to:
when in proximity to a head of the subject, operate the motor to rotate the first magnetic source causing the first magnetic source to produce a first magnetic field,
wherein the first magnetic field is applied to the second magnetic source and causes the second magnetic source to rotate and produce a second magnetic field that is applied to the head of the subject to provide a therapeutic treatment.

68. The device of claim 67, wherein the first magnetic source and the second magnetic source are each cylindrical magnets having a height and a diameter.

69. The device of claim 68, further comprising:
a first shaft running through the first magnetic source parallel to the height of the first magnetic source; and
a second shaft running through the second magnetic source parallel to the height of the second magnetic source.

70. The device of claim 69, wherein the first shaft and the second shaft are both coupled to the frame and configured to rotate with respect to the frame.

71. The device of claim 69, wherein the first shaft is coupled to the first magnetic source such that rotation of the first shaft causes the first magnetic source to rotate and the second shaft is coupled to the second magnetic source such that rotation of the second shaft causes the second magnetic source to rotate.

72. The device of claim 71, wherein the motor is coupled to the first shaft and configured to rotate the first magnetic source by rotating the first shaft.

73. The device of claim 69, wherein the motor is configured to rotate the first magnetic source in a first direction about an axis parallel to the height of the first magnetic source.

74. The device of claim 73, wherein the second magnetic source is positioned such that the first magnetic field causes the second magnetic source to rotate in a second direction, the second direction being opposite from the first direction.

75. The device of claim 74, wherein the height of the second magnetic source is adjacent to the height of the first magnetic source.

76. The device of claim 73, wherein the second magnetic source is positioned such that the first magnetic field causes the second magnetic source to rotate in the first direction.

77. The device of claim 76, wherein an end of the second magnetic source faces an end of the first magnetic source, the end of the second magnetic source corresponding to a surface having the diameter of the second magnetic source and the end of the first magnetic source corresponding to a surface having the diameter of the first magnetic source.

78. The device of claim 73, further comprising:
a third magnetic source positioned within the frame and in proximity to the second magnetic source,
wherein the second magnetic field is applied to the third magnetic source and causes the third magnetic source to rotate and produce a third magnetic field.

79. The device of claim 78, wherein the third magnetic source is positioned such that the second magnetic field causes the third magnetic source to rotate in the first direction.

80. The device of claim 79, wherein the third magnetic source is a cylindrical magnet having a height and a diameter.

81. The device of claim 80, further comprising:
a third shaft running through the third magnetic source parallel to the height of the third magnetic source.

82. The device of claim 81, wherein the third shaft is coupled to the frame and configured to rotate with respect to the frame.

83. The device of claim 82, wherein the third shaft is coupled to the third magnetic source such that rotation of the third shaft causes the third magnetic source to rotate.

84. The device of claim 80, wherein the height of the third magnetic source is adjacent to the height of the second magnetic source.

85. The device of claim 80, wherein an end of the third magnetic source faces an end of the second magnetic source, the end of the third magnetic source corresponding to a surface having the diameter of the third magnetic source and the end of the second magnetic source corresponding to a surface having the diameter of the second magnetic source.

86. The device of claim 67, wherein the at least one memory and the at least one processor are disposed within the frame.

87. The device of claim 67, further comprising:
a cover coupled to the frame and disposed between the first and second magnetic sources and the head of the subject.

88. The device of claim 67, wherein the frame includes a curvature that enables the frame to rest on the head of the subject.

89. A system for providing treatment to a subject, comprising:
a base station including a first magnetic source;
a frame configured to be disposed on a head of the subject;
a second magnetic source positioned within the frame;
at least one memory storing computer-executable instructions; and
at least one processor for executing the instructions stored on the memory, wherein execution of the instructions causes the at least one processor to:
when in proximity to a head of the subject, control the first magnetic source to produce a first magnetic field,
wherein the first magnetic field is applied to the second magnetic source and causes the second magnetic source to rotate and produce a second magnetic field that is applied to the head of the subject to provide a therapeutic treatment.

90. The system of claim 89, wherein the first magnetic field causes the second magnetic source to rotate and produce the second magnetic field when the frame is positioned adjacent to the base station.

91. The system of claim 89, wherein the first magnetic source is a stationary magnetic source.

92. The system of claim 89, wherein the first magnetic source is an electromagnetic coil.

93. The system of claim 92, wherein controlling the first magnetic source to produce the first magnetic field includes providing a current to the first magnetic to produce the first magnetic field.

94. The system of claim 89, wherein the therapeutic treatment includes transcranial magnetic stimulation (TMS).

95. The system of claim 89, wherein the first magnetic source and the second magnetic source are permanent magnets.

96. The system of claim 89, wherein the first magnetic source and the second magnetic source are each cylindrical magnets having a height and a diameter.

97. The system of claim 89, wherein the base station includes a motor coupled to the first magnetic source.

98. The system of claim 97, wherein controlling the first magnetic source to produce the first magnetic field includes operating the motor to rotate the first magnetic source.

99. The system of claim 97, wherein the motor is configured to rotate the first magnetic source about an axis parallel to the height of the first magnetic source.

100. The system of claim 97, wherein the at least one processor is configured to operate the motor.

101. The system of claim 89, further comprising:
a third magnetic source positioned within the frame and in proximity to the second magnetic source,
wherein the second magnetic field is applied to the third magnetic source and causes the third magnetic source to rotate and produce a third magnetic field.

102. A method of treating a subject, comprising:
providing a base station including a first magnetic source;
providing a frame including a second magnetic source;
disposing the frame on a head of the subject;
positioning the second magnetic source of the frame in proximity to the first magnetic source of the base station;
controlling the first magnetic source to produce a first magnetic field; and
applying the first magnetic field to the second magnetic source and causing the second magnetic source to rotate and produce a second magnetic field that is applied to the head of the subject to provide a therapeutic treatment.

103. The method of claim 102, wherein positioning the second magnetic source of the frame in proximity to the first magnetic source of the base station includes positioning the frame to be adjacent to the base station.

104. The method of claim 102, wherein the first magnetic source is a stationary magnetic source.

105. The method of claim 102, wherein the first magnetic source is an electromagnetic coil.

106. The method of claim 105, wherein controlling the first magnetic source to produce the first magnetic field includes providing a current to the first magnetic to produce the first magnetic field.

107. The method of claim 102, wherein the therapeutic treatment includes transcranial magnetic stimulation (TMS).

108. The method of claim 102, wherein the first magnetic source and the second magnetic source are permanent magnets.

109. The method of claim 102, wherein the first magnetic source and the second magnetic source are each cylindrical magnets having a height and a diameter.

110. The method of claim 102, wherein the base station includes a motor coupled to the first magnetic source.

111. The method of claim 110, wherein controlling the first magnetic source to produce the first magnetic field includes operating the motor to rotate the first magnetic source.

112. The method of claim 110, wherein the motor is configured to rotate the first magnetic source about an axis parallel to the height of the first magnetic source.

113. The method of claim 102, further comprising:
providing a third magnetic source positioned within the frame in proximity to the second magnetic source; and
applying the second magnetic field to the third magnetic source and causing the third magnetic source to rotate and produce a third magnetic field.
